(19) United States
(12) Patent Application Publication
(10) Pub. No.: US 2011/0183408 A1
(43) Pub. Date: Jul. 28, 2011

(54) REAGENT CARTRIDGE FOR MICROORGANISM DETECTION APPARATUS

(75) Inventors: Noe Miyashita, Tokyo (JP); Koji Seo, Tokyo (JP)

(73) Assignee: Hitachi Plant Technologies, Ltd.

(21) Appl. No.: 13/014,271

(22) Filed: Jan. 26, 2011

(30) Foreign Application Priority Data
Jan. 27, 2010 (JP) 2010-015662

(51) Int. Cl. C12M 1/34

(52) U.S. Cl. 435/287.1

(57) ABSTRACT

A reagent cartridge for a microorganism detection apparatus includes a plurality of reagent vessels, a support plate, and side plates, wherein the reagent vessels are integrally connected with each other in parallel by the support plate, and a group of the plurality of the reagent vessels is surrounded by the side plates; the reagent cartridge further includes an independent reagent vessel instead of at least one of the plurality of the reagent vessels and separately therefrom; and an engagement portion, wherein the engagement portion engages the independent reagent vessel in the engagement portion so as to be universally attached and detached, and to be in parallel with the plurality of the reagent vessels.

### BACKGROUND OF THE INVENTION

1. Field of the Invention

The present invention relates to a reagent cartridge for a microorganism detection apparatus.

2. Description of the Related Art

Conventionally, as a microorganism detection apparatus for detecting microorganisms in a sample, the apparatus for applying an Adenosine Tri Phosphate (hereinafter referred to as ATP) method of using ATP and for counting the microorganisms is known (for example, see Japanese Patent Laid-Open Publication No. 2008-249628).

This microorganism detection apparatus is configured to count microorganisms, depending on a luminescence intensity, when an ATP extraction liquid containing ATP that is extracted from the microorganisms in a sample is made to react with an ATP luminescence reagent in a reaction vessel.

According to such a microorganism detection apparatus, it is possible to shorten a detection time within several hours, for example, in contrast that a counting method of counting collected microorganisms according to the number of colonies cultured in a flat plate takes a few days on or before obtaining a result of the counting.

In this connection, as such a microorganism detection apparatus (for example, see Japanese Patent Laid-Open Publication No. 2008-249628), the apparatus is thought of that further comprising a mechanism of performing a process of extracting ATP from microorganisms in a sample in addition to a configuration comprising a dispense mechanism for dispensing an ATP extraction liquid in a reaction vessel in which an ATP luminescence reagent is put.

According to the microorganism detection apparatus, since the detection of microorganisms is automatically performed by providing a sample containing the microorganisms, it may be predicted to further shorten time on or before a detection result the microorganisms being obtained.

On one hand, in order to extract ATP from microorganisms in a sample, it is necessary to further comprise a process of erasing ATP existing outside a cell of a microorganism contained in the sample and a process of extracting ATP existing inside the microorganism. That is, at least an ATP erasure reagent, an ATP extraction reagent, and the ATP luminescence reagent are adapted to be arranged in the microorganism detection apparatus.

Then in order that the microorganism count apparatus is automated as described above, it is necessary, not to mention, that a dispense mechanism for dispensing these reagents in order defined in advance, and it is necessary that positions (coordinates) of the reagents are stored in a control portion of the dispense mechanism so that the dispense mechanism dispenses each of the reagents in its order.

Consequently, as a configuration for positioning these reagents, for example, the configuration can be thought of where reagent vessels of a plurality of Eppendorf (registered trademark) test tubes and the like are arranged and rested against a rack provided at predetermined positions in the microorganism detection apparatus.

However, the configuration of each of a plurality of the reagent vessels rested against the rack individually is extremely troublesome, for example, in arranging and removing the reagent vessels, and resultingly takes time additionally on or before the detection of the microorganisms.

### SUMMARY OF THE INVENTION

A problem of the present invention is to provide a reagent cartridge for a microorganism detection apparatus that enables a detection of microorganisms to be performed in shorter time.

In the reagent cartridge for the microorganism detection apparatus of the invention for solving the problem, a plurality of reagent vessels are integrally connected with each other in parallel.

According to the invention, it is possible to provide the reagent cartridge for the microorganism detection apparatus that enables the detection of microorganisms to be performed in shorter time.

### BRIEF DESCRIPTION OF THE DRAWINGS

FIG. 1 is a drawing illustrating a configuration of a microorganism detection apparatus where a reagent cartridge relating to an embodiment of the present invention is mounted.

FIG. 2 is a perspective view showing an appearance in the vicinity of amount portion of the reagent cartridge in the microorganism detection apparatus of FIG. 1.

FIG. 3 is a section drawing showing an appearance of a microorganism collector mounted on the microorganism detection apparatus of FIG. 1.

FIG. 4 is a flowchart showing processes of the microorganism detection apparatus operating, based on instructions of a control portion.

FIGS. 5A-1 to 5A-4 are section drawings schematically showing appearances in the microorganism collector when microorganisms are detected by the microorganism detection apparatus; FIGS. 5B-1 to 5B-4 are schematic drawings enlargingly showing appearances in the vicinity of a filter corresponding to the scenes of FIGS. 5A-1 to 5A-4.

FIG. 6A is a perspective view of the reagent cartridge relating to the embodiment; FIG. 6B is a VIb-VIb section drawing in FIG. 6A.

FIG. 7A is a perspective view showing a first modification example of the reagent cartridge; FIG. 7B is a perspective view showing a second modification example of the reagent cartridge; and FIG. 7C a perspective view showing a third modification example of the reagent cartridge.

FIG. 8A is a perspective view showing a fourth modification example of the reagent cartridge and an appearance of the reagent cartridge relating to the fourth modification example being mounted on the microorganism detection apparatus; FIGS. 8B and 8C are concept drawings showing appearances of the reagent cartridge of the fourth modification example being locked in the microorganism detection apparatus when the reagent cartridge is mounted on the apparatus, and are the drawings corresponding to a VIII-VIII section in FIG. 8A.

### BEST MODE(S) FOR CARRYING OUT THE INVENTION

Next will be described a reagent cartridge relating to an embodiment of the present invention in detail with reference to drawings as needed.

Hereafter, as an example, taking a reagent cartridge attached to an apparatus for counting microorganisms in a sample (hereinafter referred to as microorganism count apparatus) which the apparatus is a microorganism detection apparatus, a general configuration of the microorganism count apparatus and a counting principle of the microorganisms in the microorganism count apparatus will be described; and then the reagent cartridge relating to the embodiment will be described.

### <General Configuration of Microorganism Count Apparatus>

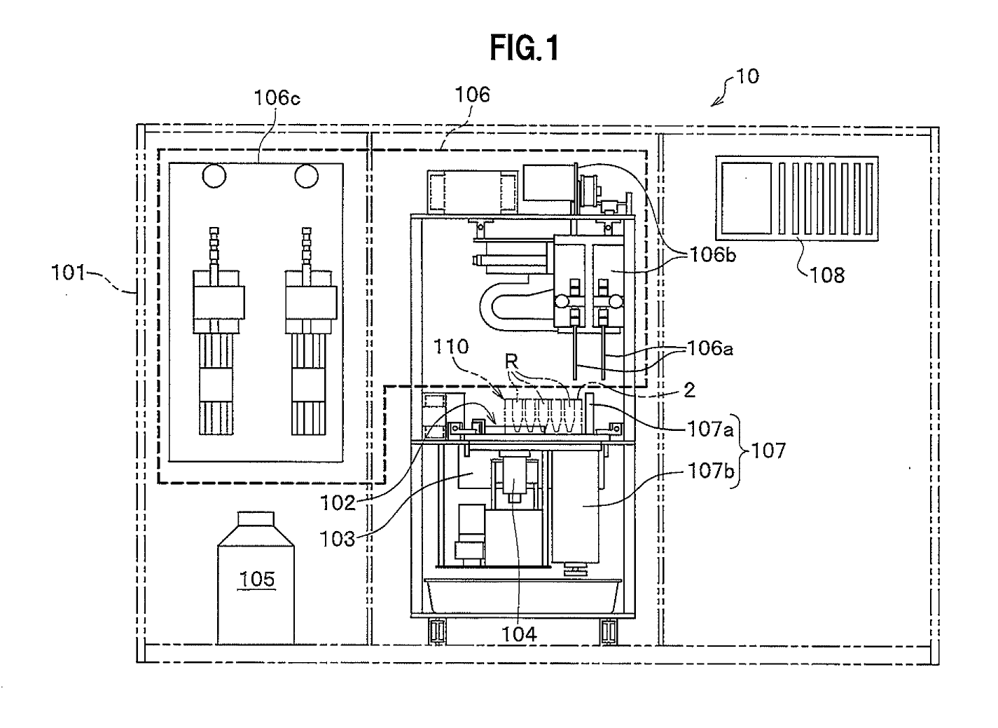

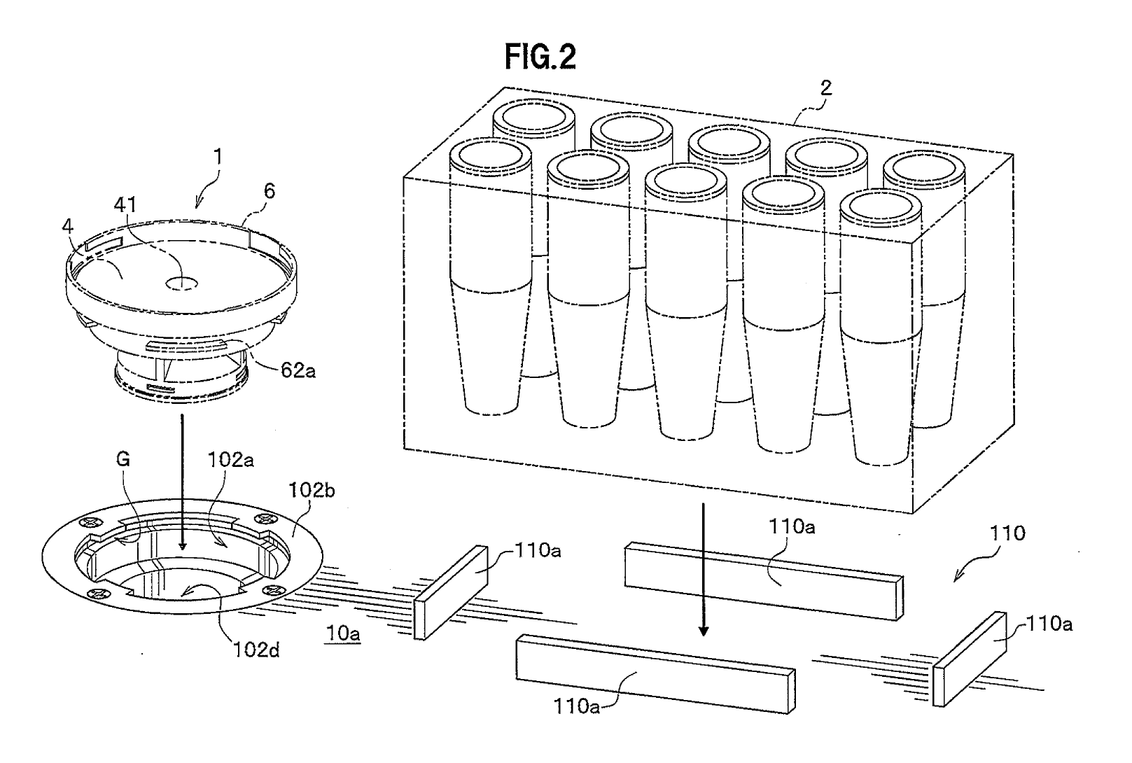

As shown in FIG. 1, a microorganism count apparatus 10 is an apparatus for counting microorganisms contained in a sample in conformity with an ATP method, and comprises a reagent mount portion 110 configured to mount a reagent cartridge 2 where a plurality of reagents R required for performing the ATP method are contained in a casing 101; a sample mount portion 102 configured to mount a microorganism collector 1 (see FIG. 2) having the sample therein; a hot water supply portion 103; a suction unit 104; a liquid tank 105; a dispense unit 106; a luminescence intensity measurement unit 107; and a control portion 108.

In addition, in FIG. 1 the casing 101 and the reagent cartridge 2 are shown in virtual lines for convenience' sake of drawing thereof, and the microorganism collector 1 is omitted.

The reagent mount portion 110 is configured, as shown in FIG. 2, with lock portions 110a arranged so as to position the reagent cartridge 2 at a predetermined position of an apparatus main body 10a. The lock portions 110a of the embodiment comprise four ribs vertically provided at the apparatus main body 10a; the four ribs abut with a lower side of the reagent cartridge 2, which will be described later, from four directions, and thereby can attachably and detachably lock the reagent cartridge 2 on the apparatus main body 10a.

The sample mount portion 102 comprises, as shown in FIG. 2, a depression 102a configured to accommodate a housing 6, which will be described later, of the microorganism collector 1; around an opening of the depression 102a is further arranged an engagement ring 102b.

The engagement ring 102b engages with engagement claws 62a and thereby is adapted to support the housing 6 at the sample mount portion 102. In this connection, the engagement ring 102b comprises notches 102d like a flat shape so that the engagement claws 62a of the housing 6 are housed, and forms gaps G in a thickness receivable the claws 62a between itself and the apparatus main body 10a where the depression 102a is formed.

That is, in fitting the housing 6 in the depression 102a, the claws 62a are inserted in the depression 102a through the notches 102d, the housing 6 is turned, the claws 62a are slid in the gaps G, and thereby the housing 6 is adapted to engage with the engagement ring 102b.

The microorganism collector 1 mounted on the sample mount portion 102 thus described comprises, as shown in FIG. 2, the housing 6 presenting a funnel form like an approximately reverse cone, and a collection dish 4 placed so as to close an upper opening of the housing 6.

The collection dish 4 presents a disc form, and in its middle portion is formed a through-hole 41 penetrating the dish 4 up and down.

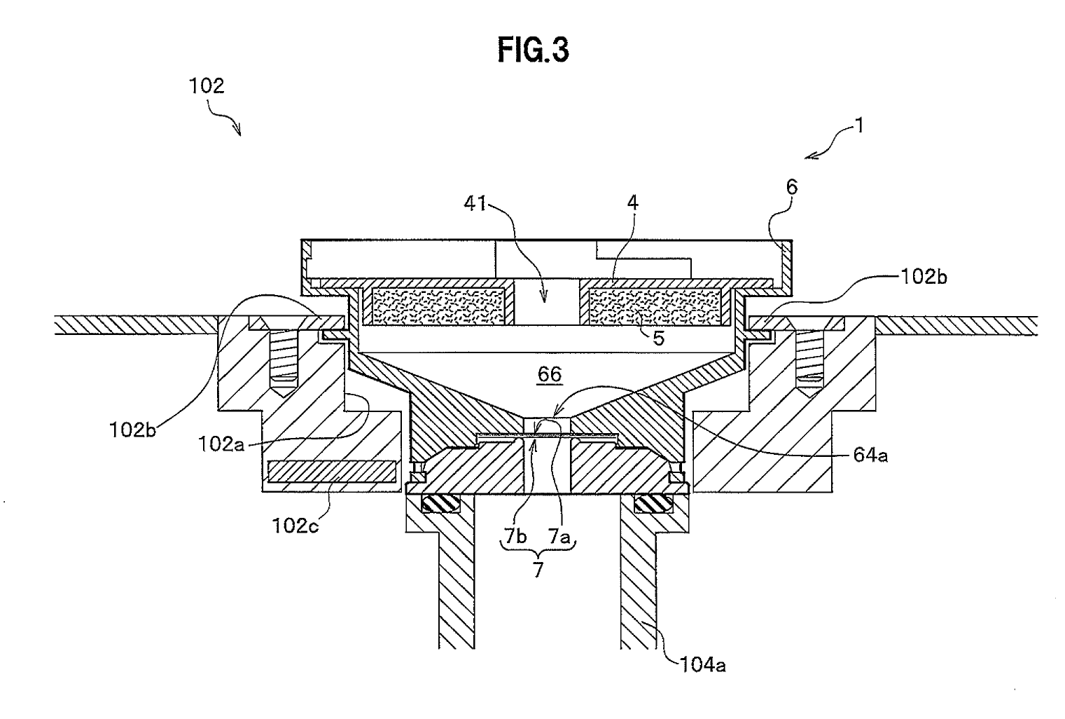

Then, as shown in FIG. 3, with respect to the microorganism collector 1 mounted on the sample mount portion 102, the through-hole 41 opens upwards, and an internal space 66 and outside of the housing 6 are communicated.

Furthermore, a carrier 5 is arranged on a backside of the collection dish 4. The carrier 5 is for receiving an air flow sucked by an air sampler (not shown) and collecting microorganisms accompanied with the air when the collection dish 4 is arranged at the air sampler so that the carrier 5 is upward.

In this connection, the carrier 5 of the embodiment is formed of a material changing in phase from gel to sol by being increased in temperature from a normal temperature. As the material of the carrier 5, such a material is preferable that changes in phase to sol at not less than 30 degrees Celsius, and more preferable that is liquefied between 37 and 40 degrees Celsius. Above all are preferable gelatine, a mixture of gelatine and glycerol, and a copolymer of a ratio of 10 to 1 of N-acryloyl glycinamide and N-methacryloyl-N′-biotinyl propylene diamine.

Then at a lowest portion of the housing 6 is formed, as shown in FIG. 3, a discharge opening 64a configured to discharge a content of the internal space 66; at an exit of the opening 64a is arranged a filter 7. The filter 7 is a membrane filter, has a double structure, and a hydrophilic filter 7a and a hydrophobic filter 7b arranged respectively on an upper side and a lower side are superimposed.

Additionally, in FIG. 3 a reference symbol 102a shows the depression described above and configuring the sample mount portion 102; a reference symbol 102b shows the engagement ring configured to be engaged with the engagement claws 62a of the housing 6; and a reference symbol 104a shows a suction head configuring the suction unit 104 described later.

Furthermore, in FIG. 3 a reference symbol 102c shows a heater embedded in a good conductive member (for example, aluminum member and the like) arranged around the depression 102a. The heater 102c increases the gel-like carrier 5 in temperature and promotes it to become gel when the microorganism collector 1 is mounted on the sample mount portion 102.

The hot water supply portion 103 shown in FIG. 1 warms up sterilized distilled water supplied from the liquid tank 105 and supplies the water. Further describing the hot water supply portion 103 in more detail, the portion 103 instills the hot water in the internal space 66 (see FIG. 3) of the housing 6 through the through-hole 41 (see FIG. 3) of the collection dish 4. As the hot water supply portion 103, although not shown, such a supply portion can be cited, for example, that discharges hot water through a nozzle formed of a flexible tube and the like, wherein the hot water is warmed up to a predetermined temperature, while being sent, by warming up a piping tube connected to a peristaltic pump by means of a tube heater, a cartridge heater, and the like. In this connection, the nozzle is inserted in advance in the internal space 66 (see FIG. 3) of the housing 6 through the through-hole 41 of the collection dish 4 in mounting the microorganism collector 1 on the sample mount portion 102.

The suction unit 104 shown in FIG. 1 sucks the hot water instilled in the internal space 66 (see FIG. 3) of the housing 6, the reagents R described later, and the like, and thereby discharges these through the filter 7 (see FIG. 3). The suction unit 104 can be configured, for example, with the suction head 104a (see FIG. 3), a suction pump and an effluent tank both not shown and connected to the head 104a through a predetermined piping, and the like.

In this connection, the suction unit 104 of the embodiment further comprises an elevating device (not shown) for moving the suction head 104a (see FIG. 3) up and down so that the head 104a can be connected and disconnected with respect to the housing 6 (see FIG. 3) supported at the sample mount portion 102.

The liquid tank 105 shown in FIG. 1 reserves a liquid such as sterilized distilled water and a buffer (for example, phosphate solution). The liquid tank 105 of the embodiment is assumed, as described above, to reserve sterilized distilled water. The sterilized distilled water, for example, is added in the housing 6 for any of enhancing a filtration speed of the carrier 5 (see FIG. 3) of the microorganism collector 1 described later and washing the housing 6, and is further filled in a piping system of a syringe pump 106c of the dispense unit 106 shown in FIG. 1 and described later.

The dispense unit 106 shown in FIG. 1 dispenses the reagents R in the housing 6 of the microorganism collector 1. Furthermore, the dispense unit 106 dispenses any of the reagents R and an ATP extraction reagent described later in the housing 6 (see FIG. 3) in a luminescence tube 107a of the luminescence intensity measurement unit 107.

The dispense unit 106 may comprise such dispense nozzles 106a formed of each thin pipe, actuators 106b configured to move the nozzles 106a in three-axis directions, the syringe pump 106c connected to the nozzles 106a by a predetermined flexible piping, and a piping (not shown) for supplying sterilized distilled water to the nozzles 106a through the syringe pump 106c

As the luminescence intensity measurement unit 107 shown in FIG. 1, such a measurement unit can be cited that comprises the luminescence tube 107a configured to receive an ATP extraction liquid (described later) dispensed from an inside of the housing 6 (see FIG. 3) and to make ATP emit light, and a photon-detection-portion main body 107b having such a photomultiplier tube (PMT) configured to detect a luminescence intensity of the ATP.

The control portion 108 shown in FIG. 1 is configured so as to overall and generally control the microorganism count apparatus 10, to mount the microorganism collector 1 (see FIG. 3) on the sample mount portion 102, and then to control the hot water supply portion 103, the suction unit 104, the dispense unit 106, and the luminescence intensity measurement unit 107 according to a procedure described next. The control portion 108 thus described comprises a CPU (Central Processing Unit), a ROM (Read Only Memory), a RAM (Random Access Memory), various interfaces, an electronic circuit, and the like.

### <Operation of Microorganism Count Apparatus and Counting Principle of Microorganisms>

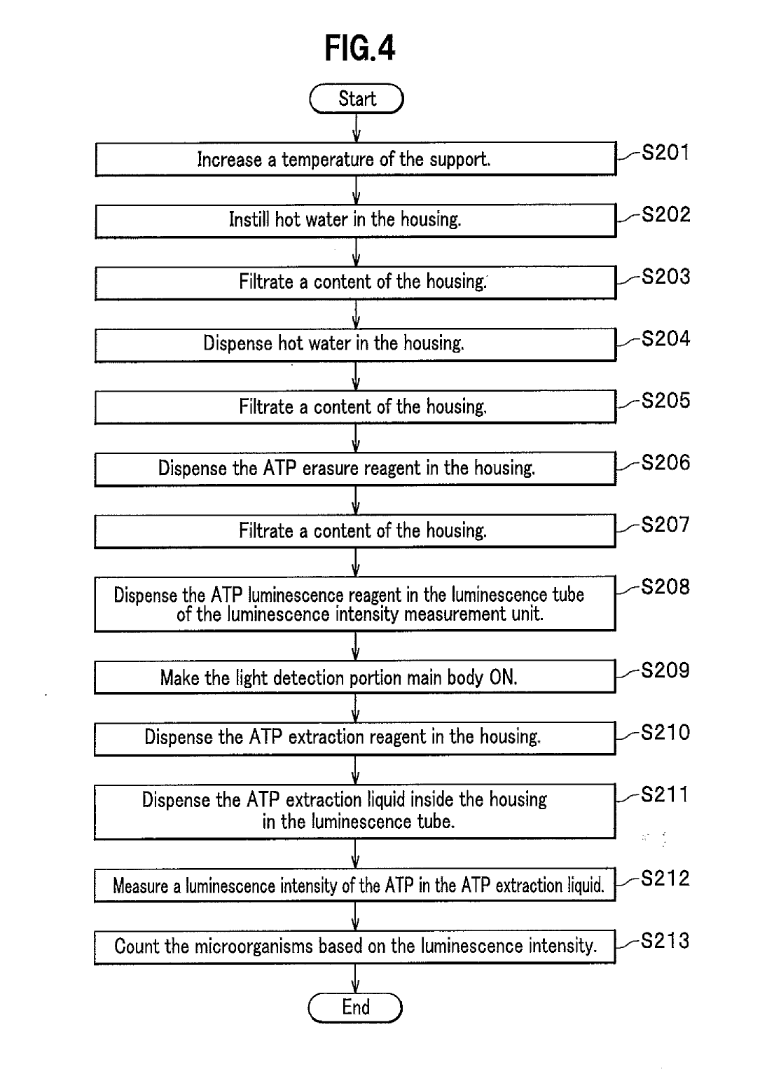

Next, while describing a procedure performed by the control portion 108, an operation of the microorganism count apparatus 10 and a counting principle of microorganisms will be described with reference to FIG. 4.

According to the microorganism count apparatus 10 shown in FIG. 1, after the microorganism collector 1 (see FIG. 3) is mounted on the sample mount portion 102, an activation switch not shown is made ON, and thereby the control portion 108 performs a next procedure.

The control portion 108 outputs, as shown in FIG. 4, an instruction to a predetermined inverter and the like so as to turn on electricity to the heater 102c (see FIG. 3) and makes the heater 102c generate heat. That is, the control portion 108 makes the carrier 5 (see FIG. 3) of the microorganism collector 1 increased in temperature by the heater 102c (step S201). As a result thereof, the carrier 5 is made into a state of sol, and thereby peeled off onto a bottom portion in the housing 6 (see FIG. 3) from the collection dish 4 (see FIG. 3)

Next, the control portion 108 outputs an instruction to the hot water supply portion 103 (see FIG. 1) and makes the portion 103 instill hot water in the housing 6 (see FIG. 3) (step S202). As a result thereof, the carrier 5 (see FIG. 3) is promoted to be sol and is diluted by the hot water.

Next, the control portion 108 outputs an instruction to the suction unit 104 (see FIG. 1), makes the unit 104 connect the suction head 104a (see FIG. 3) to the housing 6 (see FIG. 3) and suck the housing 6, and filtrate a content of the housing 6 (step S203). As a result thereof, microorganisms collected in the carrier 5 is separated by the filter 7 (see FIG. 3) and held, and the diluted carrier 5 is filtrated and discharged outside the housing 6.

Next, the control portion 108 again outputs an instruction to the hot water supply portion 103 and makes the portion 103 dispense hot water in the housing 6 (see FIG. 3) (step S204). After that, the hot water is again filtrated (step S205). Thus the inside of the housing 6 is washed and a recovery ratio of microorganisms is enhanced.

Next, the control portion 108 outputs an instruction to the dispense unit 106 (see FIG. 1) and makes the unit 106 dispense an ATP erasure reagent (reagents R in FIG. 1) of the reagent cartridge 2 (step S206). As a result thereof, ATP existing outside cells of microorganisms on the filter 7 is erased.

Next, the control portion 108 outputs an instruction to the suction unit 104 (see FIG. 1), and makes the unit 104 suck the housing 6 (see FIG. 3) and filtrate a content thereof (step S207). As a result thereof, microorganisms are separated by the filter 7 (see FIG. 3) and held, and the ATP erasure reagent and the hot water are filtrated and discharged outside the housing 6.

Next, the control portion 108 outputs an instruction to the dispense unit 106 (see FIG. 1) and makes the unit 106 dispense an ATP luminescence reagent (reagents R in FIG. 1) of the reagent cartridge 2 in the luminescence tube 107a (see FIG. 1) (Step S208).

Next, the control portion 108 outputs an instruction to the luminescence intensity measurement unit 107 (see FIG. 1) and makes the photon-detection-portion main body 107b (see FIG. 1) ON by the unit 107 (step S209).

Next, the control portion 108 outputs an instruction to the dispense unit 106 (see FIG. 1) and makes the unit 106 dispense an ATP extraction reagent (reagents R in FIG. 1) of the reagent cartridge 2 in the housing 6 (see FIG. 3) (step S210). As a result thereof, ATP is extracted from the microorganisms held by the filter 7 and a sample liquid is adjusted on the filter 7.

As a result of steps S208 and S209, a light detection portion of the luminescence intensity measurement unit 107 measures a background of the ATP luminescence reagent in the luminescence tube 107a.

Next, the control portion 108 outputs an instruction to the dispense unit 106 (see FIG. 1) and makes the unit 106 dispense an ATP extraction liquid EX in the housing 6 into the luminescence tube 107a for which the background is measured (step S211). As a result thereof, in the luminescence tube 107a, light is emitted due to a reaction between the ATP extraction liquid EX and the ATP luminescence reagent dispensed first in the step S208

Next, the control portion 108 processes a signal into a digital form, wherein the photon-detection-portion main body 107b (see FIG. 1) of the luminescence intensity measurement unit 107 (see FIG. 1) detects a luminescence of the ATP and outputs the signal, and the portion 108 measures a luminescence intensity of the ATP, based on a single photon counting method (step S212). Then the control portion 108 calculates an ATP quantity (amol) contained in the ATP extraction liquid EX dispensed in the luminescence tube 107a, based on a working curve showing a relationship between an ATP quantity (amol) stored in advance and a luminescence intensity (CPS: Count Per Second), and counts microorganisms (step S213) as an ATP conversion value equivalent to the microorganisms contained in the carrier 5, based on the ATP quantity (amol) and an ATP extraction liquid quantity in the sample liquid adjusted in the step S210.

When the microorganism count apparatus 10 is thus operated, a state in the microorganism collector 1 will be described with reference to FIGS. 5A-1 to 5A-4 and FIGS. 5B-1 to 5B-4.

In addition, in FIGS. 5B-1 to 5B-4 microorganisms shown by reference symbols B are actually sizes in a micrometer level; ATP is actually a size of a molecule level; and FIGS. 5B-1 to 5B-4 do not indicate these relative sizes.

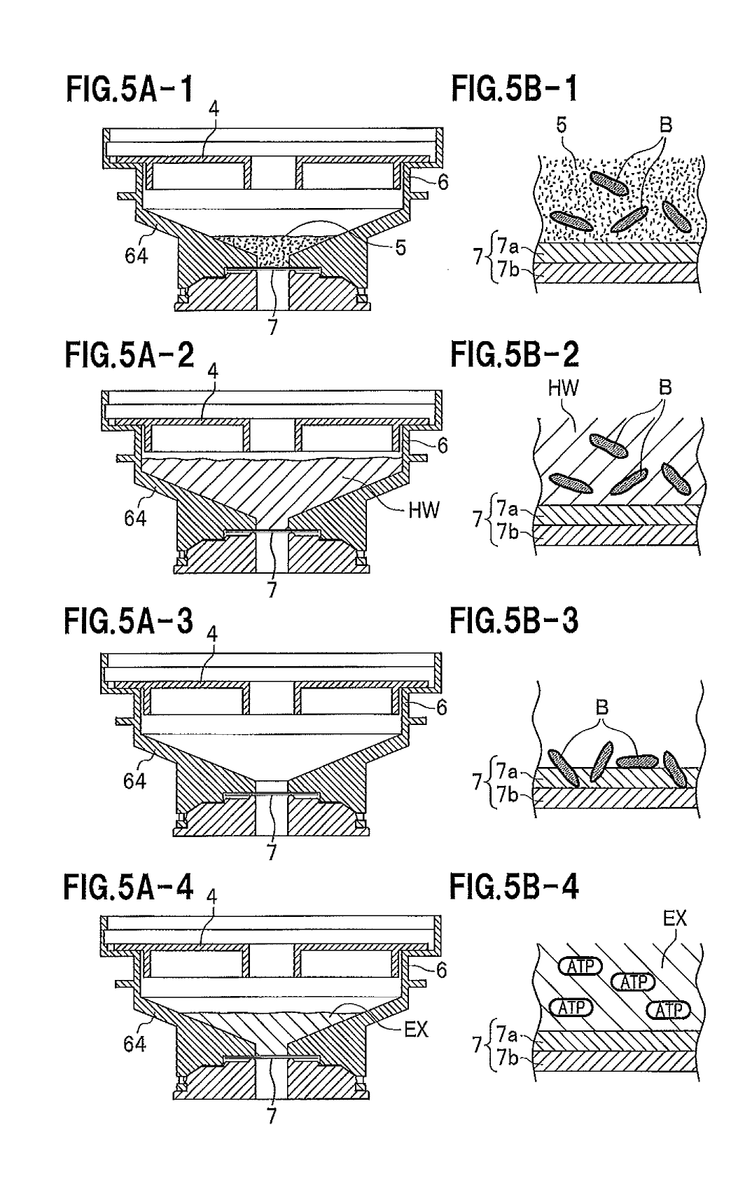

In the step S201 (see FIG. 4), when the carrier 5 is increased in temperature, as shown in FIG. 5A-1, the carrier 5 held in the collection dish 4 becomes sol and is peeled off on the funnel portion 64 of the housing 6. At this moment as shown in FIG. 5B-1, the microorganisms B collected in the carrier 5 stay with it on the filter 7.

Next, in the step S202 (see FIG. 4), when hot water HW is instilled in the housing 6, the carrier 5 is further promoted to be sol and diluted by the water HW. At this moment, with respect to the filter 7, because the lower side thereof is configured with the hydrophobic filter 7b as shown in FIG. 5A-2, the hot water HW diluting and containing the carrier 5 stays in the housing 6. In addition, in FIG. 5A-2, a reference symbol 4 indicates the collection dish; a reference symbol 64 indicates the funnel portion

Next, in the step S203 (see FIG. 4), when the content of the housing 6 is filtrated, the hot water HW diluting and containing the carrier 5 (see FIG. 5A-2) is discharged as shown in FIG. 5A-3. At this moment, the microorganisms B in the hot water HW diluting and containing the carrier 5 are separated by the filter 7 and held as shown in FIG. 5B-3.

In this connection, since the filter 7 of the embodiment is the double structure of the hydrophilic filter 7a and the hydrophobic filter 7b as shown in FIG. 5B-3, the filter 7 can hold a liquid at an upper portion thereof by an action of the hydrophobic filter 7b, as far as a suction filtration or a pressure filtration is not applied, different from a filter formed only of a hydrophilic filter used in a conventional ATP method; therefore, it is possible to perform a reagent reaction process such as an ATP extraction on the filter 7.

Then after the ATP erasure reagents are dispensed in the housing 6 in the step S206 (see FIG. 4), the ATP extraction reagent is dispensed in the housing 6 in the step S210 (see FIG. 4).

Then in the step S210 (see FIG. 4), as shown in FIG. 5A-4, an ATP extraction liquid EX stays in the housing 6. At this moment, as shown in FIG. 5B-4, the ATP exists in the ATP extraction liquid EX in a quantity corresponding to the number of the microorganisms B.

Then in the step S211 (see FIG. 4), the ATP extraction liquid EX shown in FIG. 5B-4 is dispensed in the luminescence tube 107a (see FIG. 1), and at this moment, microorganisms are counted according to a luminescence intensity then.

### <Reagent Cartridge>

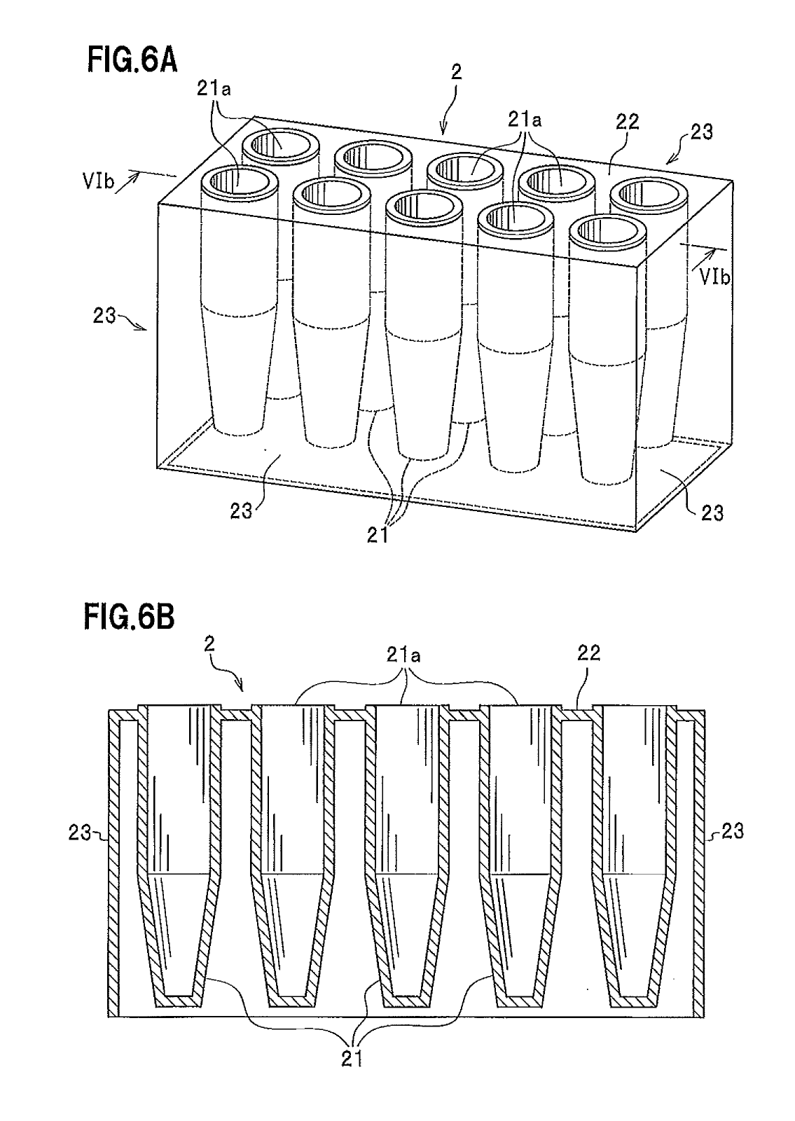

Next will be described the reagent cartridge 2 of the embodiment in more detail with reference to FIGS. 6A and 6B.

As shown in FIGS. 6A and 6B, the reagent cartridge 2 comprises a plurality of reagent vessels 21 configured with each tube-like body having a bottom. Each of the reagent vessels 21 comprises an opening 21a at an upper portion thereof; a bottom portion of each of the vessels 21 presents approximately a reverse cone and is gradually reduced in diameter.

In this connection, with respect to the reagent cartridge 2 of the embodiment, total ten reagent vessels 21 are arranged in parallel in two columns and five rows at an equal interval.

The reagent vessels 21 are integrally connected with each other through peripheral walls of the vessels 21 by a support plate 22 whose profile is rectangular in plan view. Particularly, in the embodiment the support plate 22 integrally connects the reagent vessels 21 with each other through the peripheral walls of the vessels 21 in the vicinity of the openings 21a of the vessels 21.

Then with respect to the reagent cartridge 2, a group of the ten reagent vessels 21 is surrounded by four rectangular side plates 23 respectively connected to four edges of the support plate 22, a contour of the cartridge 2 is formed into an approximate cuboid.

The reagent cartridge 2 can be molded by a moldable resin, and above all, PP (polypropylene) is preferable.

With respect to the reagent cartridge 2 thus described, a plurality of the reagents R required in the ATP method, specifically, the ATP erasure reagent, the ATP extraction reagent, and the ATP luminescence reagent are put in each of the reagent vessels 21.

In this connection, as the ATP erasure reagent, for example, an ATP decomposition enzyme can be cited.

As the ATP extraction reagent, for example, benzalkonium chloride, trichloroacetic acid, a tris buffer, and the like can be cited.

As the ATP luminescence reagent, for example, a luciferase-luciferin reagent can be cited.

Furthermore, in these reagents R can be included such a plurality of ATP reagents diluted gradually to a known concentration in order to make a working curve indicating a relationship between the ATP quantity (amol) and the luminescence intensity (CPS); a correction reagent (ATP standard reagent) of the luminescence intensity measurement unit 107 (see FIG. 1); a correction reagent (ATP standard reagent) when an preserved ATP luminescence reagent and the like are used; and sterilized pure water.

Then these reagents R are put in each of the reagent vessels 21; thereby each of the reagents R is in one lump, and is positioned and arranged at the reagent mount portion 110 in the vicinity of the microorganism collector 1 mounted on the sample mount portion 102, preferably in the vicinity of the collector 1 and the luminescence reagent tube 107a (see FIG. 1).

That is, with respect to the reagent cartridge 2, each of the reagents R is positioned so that the dispense nozzles 106a can dispense, by their shortest distance movement, each of the reagents R in a predetermined order in the housing 6 of the microorganism collector 1 and in the luminescence tube 107a of the luminescence intensity measurement unit 107.

In this connection, the position (coordinates) of each of the reagents R is stored in the control portion 108 for controlling the dispense unit 106.

Next will be described an action and effect of the reagent cartridge 2 of the embodiment.

According to the reagent cartridge 2 thus described, since the plurality of the reagent vessels 21 are integrally connected with each other, the plurality of the reagents R are put in each of the vessels 21 required in the ATP method; thereby, it is possible to aggregate the reagents R in one lump.

That is, a user can arrange the plurality of the reagents R at predetermined positions (coordinates) thereof, respectively, which the control portion 108 for controlling the dispense unit 106 refer to, by one simple process operation of merely arranging the reagent cartridge 2 at the reagent mount portion 110.

Thus, according to the reagent cartridge 2, because it is possible to easily arrange the reagent cartridge 2 at the microorganism count apparatus 10 and remove the cartridge 2 therefrom, for example, different from a configuration of individually arranging a plurality of reagent vessels and resting the vessels against a rack provided at a predetermined position in the microorganism detection apparatus 10, it is resultingly possible to shorten time on or before microorganisms being detected.

Furthermore, according to the reagent cartridge 2, user's hand and fingers do not touch all of the reagent vessels 21, for example, different from the configuration of individually arranging a plurality of reagent vessels and resting the vessels against a rack provided at a predetermined position in the microorganism detection apparatus 10.

Particularly, by a user gripping the side plates 23 of the reagent cartridge 2, it is possible to completely avoid user's hand and fingers to touch the reagent vessels 21.

Thus, according to the reagent cartridge 2, it is possible to more surely prevent any of the reagent vessels 21 and the reagents R from being contaminated by a substance that is a disturbance factor of a microorganism detection, and resultingly to more accurately detect microorganisms contained in the sample.

Furthermore, with respect to the reagent cartridge 2 because the plurality of the reagent vessels 21 arranged at the equal intervals by the support plate 22 and the side plates 23 support themselves, and the plate 22 and the plates 23 are arranged so as to surround the vessels 21, the cartridge 2 can be manufactured to be more lightweight and at a low cost, for example, in comparison with the plurality of the reagent vessels 21 whose spacing between each thereof is formed to be solid.

Furthermore, with respect to the reagent cartridge 2, since the plurality of the reagent vessels 21 arranged at the equal intervals by the support plate 22 and the side plates 23 support themselves, and the plate 22 and the plates 23 are arranged so as to surround the vessels 21, it is possible to ensure a contact area between each of the vessels 21 and an atmosphere to be large.

Thus, according to the reagent cartridge 2, when refrigerated reagents R are instilled, the reagents R can be returned earlier to a room temperature.

Furthermore, with respect to the reagent cartridge 2, since the plurality of the reagent vessels 21 arranged at the equal intervals by the support plate 22 and the side plates 23 support themselves, and the plate 22 and the plates 23 are arranged so as to surround the vessels 21, it is possible to easily perform die cutting in molding the cartridge 2.

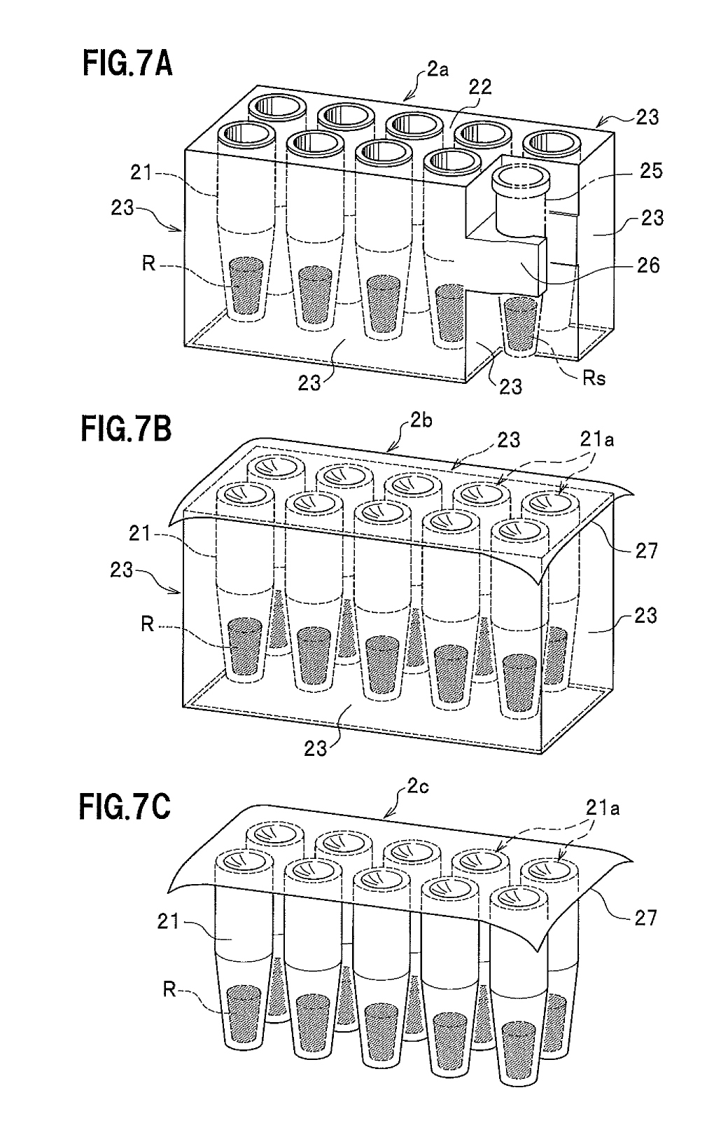

Although the embodiment of the present invention is thus described, the invention is not limited thereto and can be embodied by various modes, for example, as shown in FIGS. 7A to 7C.

As shown in FIG. 7A, a reagent cartridge 2a of a first modification example comprises an engagement portion 26 configured to engage an independent reagent vessel 25 in the cartridge 2a so that the vessel 25 is universally attached to and detached from the cartridge 2a, wherein the vessel 25 is separate from a plurality of reagent vessels 21 integrated.

Furthermore, with respect to the reagent cartridge 2a, one reagent vessel 21 positioned at one corner of the reagent cartridge 2 of the embodiment is omitted, and instead of the omitted reagent vessel 21, the engagement portion 26 is provided so that the independent reagent vessel 25 is arranged so as to be universally attached to and detached from the cartridge 2a.

The reagent cartridge 2a is configured so that the engagement portion 26 extended from side plates 23 (see FIGS. 6A and 6B) forming the corner, where the reagent vessel 21 is omitted, grips a peripheral wall of the independent reagent vessel 25 by an elastic force of the portion 26.

According to the reagent cartridge 2a of the first modification example thus described, an effect similar to that of the reagent cartridge 2 of the embodiment is obtained and a reagent Rs can be separately included in the cartridge 2a, wherein the reagent Rs is adjusted in an environment (for example, by a control standard of a more severe purity) different from that of the reagents R instilled in the reagent vessels 21.

In addition, although the reagent cartridge 2a of the first modification example is configured so as to arrange the independent reagent vessel 25 instead of the reagent vessel 21 positioned at one corner being omitted, the cartridge 2a may also be configured so that the engagement portion 26 is provided at any of the side plates 23 of the reagent cartridge 2 of the embodiment, where no reagent vessel 21 is omitted, and the independent reagent vessel 25 may be arranged at any of the side plates 23.

As shown in FIG. 7B, with respect to a reagent cartridge 2b of a second modification example, predetermined reagents R are instilled in the reagent vessels 21, and the openings 21a of all the vessels 21 are closed by one sheet member 27 of an openable sealing member. The sheet member 27 is a laminate film of a thermoplastic resin film and an aluminum foil, and a thermoplastic resin film side thereof is fusion-bonded to an opening edge of the opening 21a of each of the reagent vessels 21.

In this connection, a user can peel off the sheet member 27 from a reagent vessels 21 side by her/his hand and fingers.

Furthermore, the sheet member 27 can be pierced by the dispense nozzles 106a (see FIG. 1) of the dispense unit 106.

According to the reagent cartridge 2b of the second modification example thus described, an effect similar to that of the reagent cartridge 2 of the embodiment is obtained, and while the reagents R in the reagent vessels 21 can be normally taken separately, it is possible to surely prevent the reagents R from being contaminated.

Thus, according to the reagent cartridge 2b of the second modification example, it is possible to more accurately detect microorganisms.

In addition, in the reagent cartridge 2b of the second modification example, although all the reagent vessels 21 are closed by the one sheet member 27, the cartridge 2b of the invention may include a plurality of sheet members 27 for closing openings 21a with respect to a few reagent vessels 21 or closing an opening 21a with respect to each of the reagent vessels 21.

Furthermore, a sealing member of the reagent cartridge 2b of the second modification example is not limited to the sheet member 27, and an openable stopper is also available.

As shown in FIG. 7C, a reagent cartridge 2c of a third modification example, the plurality of the reagent vessels 21 are integrally connected with each other in parallel by nothing but one sheet member 27.

According to the reagent cartridge 2c of the third modification example, an effect similar to that of the reagent cartridge 2 of the embodiment is obtained, and since the reagent vessels 21 are integrally connected by nothing but the one sheet member 27, the cartridge 2c can be manufactured to be more lightweight and at a low cost.

Additionally, the reagent cartridge 2c of the third modification example can be divided into a few reagent vessels 21 by giving a cutoff line, although not shown, such as a perforated line to the sheet member 27.

Although in the embodiment the lock portion 110a formed of the ribs abutting with the reagent cartridge 2 has been exemplified as the reagent mount portion 110, the cartridge 2 can comprise locked portions locked to the lock portion 110a of the apparatus main body 10a, wherein the locked portions are universally locked and, unlocked.

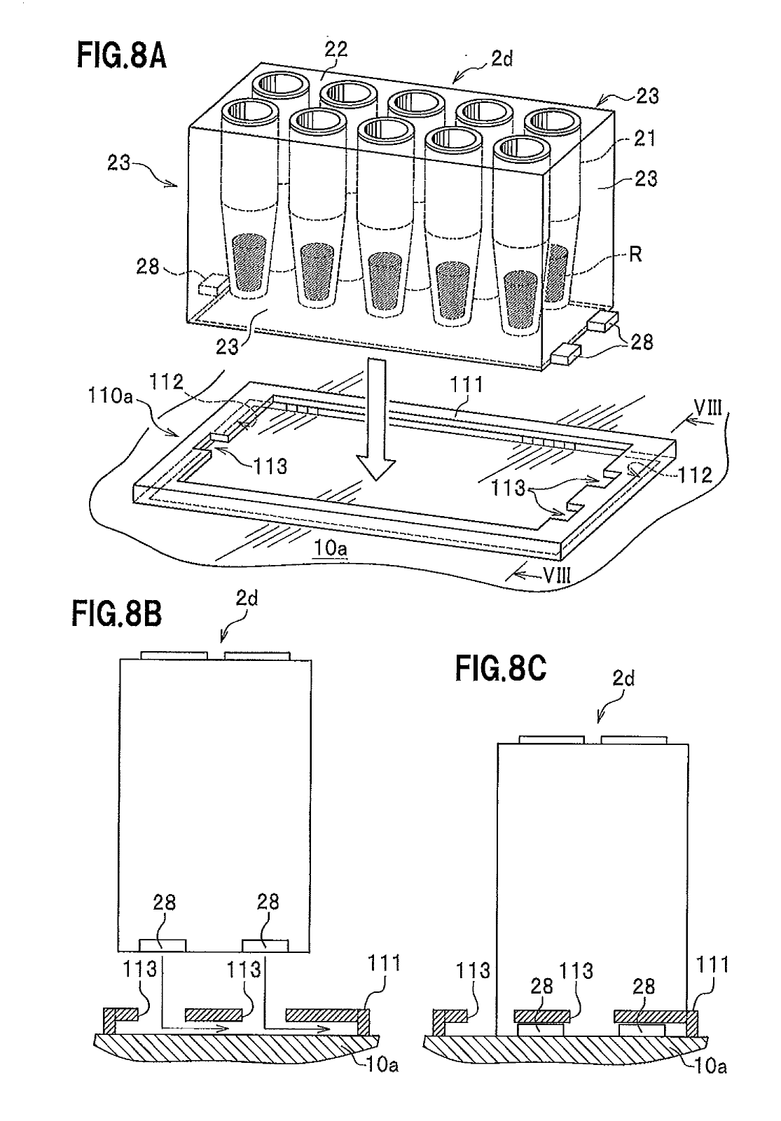

Next will be specifically described a reagent cartridge 2d (fourth modification example) comprising protrusions configured to fit in the depression (corresponding to grooves 112 described later) provided at the apparatus main body 10a with reference to FIGS. 8A and 8B. Additionally, in the fourth modification example, to elements similar to those of the reagent cartridge 2 will be appended same symbols; detailed descriptions thereof will be omitted.

As shown in FIG. 8A, the reagent cartridge 2d of the fourth modification example comprises protrusions 28 at a pair of side plates 23 out of four side plates 23, wherein the pair of the side plates 23 are connected to a shorter edge side of the support plate 22 and comprise the protrusions 28. The protrusions 28 correspond to the “locked portions.” The protrusions 28 are formed at lower edges of the pair of the side plates 23 and are protruded outwards from the plates 23. The protrusions 28 are asymmetric in the pair of the side plates 23; the protrusion 28 formed at one side plate of the pair is one, and the protrusions 28 formed at the other side plate of the pair are two. These protrusions 28 are adapted to be locked to the lock portion 110a provided at the apparatus main body 10a so as to be universally locked and unlocked.

Here, the lock portion 110a is formed of a frame body 111 configured to receive a bottom portion of the reagent cartridge 2d. On a pair of shorter edge sides of the frame body 111 are formed grooves 112, respectively, and the protrusions 28 are adapted to be able to be slidingly moved in the grooves 112 of the received reagent cartridge 2d. On the shorter edge sides of the frame body 111, notches 113 leading to the grooves 112 are formed at positions corresponding to the protrusions 28 of the reagent cartridge 2d, and the protrusions 28 are received in the grooves 112 through the notches 113, respectively.

As shown in FIGS. 8B and 8C, with respect to the reagent cartridge 2d, the protrusions 28 are fitted in the grooves 112 through the notches 113, are slidingly moved, and thereby are locked to the frame body 111 and fixed. Furthermore, the reagent cartridge 2d is moved in a route reverse to the protrusions 28, and thereby the lock of the protrusions 28 with respect to the frame body 111 is released and the cartridge 2d is removable from the body 111.

According to the reagent cartridge 2d thus described, since the protrusions 28 are fixed to the apparatus main body 10a through the frame body 111, it is possible to more surely position the cartridge 2d with respect to the main body 10a.

Furthermore, according to the reagent cartridge 2d thus described, since the protrusions 28 are asymmetric in the respective facing side plates 23, it is possible to prevent a user from mistaking an arrangement direction of the cartridge 2d. As a result thereof, the plurality of the reagents R are properly arranged at the apparatus main body 10a.

Furthermore, although the reagent cartridge 2d is configured to be asymmetric by changing the number of the protrusions 28 between one side plate 23 and the other side plate 23, the cartridge 2d may also be configured to be asymmetric by changing a form of the protrusions 28.

Furthermore, it may also be available in the present invention that the locked portion (protrusions 28) is provided at the reagent cartridge 2a having the engagement portion 26 shown in FIG. 7A and the reagent cartridge 2b having the sheet member 27 shown in FIG. 7B.

Particularly, the reagent cartridge 2b having the sheet member 27 as described above can prevent the cartridge 2b from being lifted up from the lock portion 110a when the dispense nozzles 106a (see FIG. 1) pierce the sheet member 27 and then are moved upward.

Furthermore, although the reagent cartridge 2d shown in FIGS. 8A to 8C comprises the protrusions 28 at the side plates 23 connected to the shorter edge sides of the support plate 22, such a reagent cartridge may also be available in the invention, wherein the protrusions 28 are arranged at a pair of side plates 23 connected to longer edge sides of the plate 22.

In this connection, with respect to the frame body 111 of the apparatus main body 10a configured to lock the protrusions 28 of the reagent cartridge 2d, the grooves 112 are formed on the pair of the longer edge sides of the frame body 111, respectively, and as a result thereof, the protrusions 28 of the cartridge 2d are slidingly moved along the grooves 112.

What is claimed is:

1. A reagent cartridge for a microorganism detection apparatus comprising:
a plurality of reagent vessels;
a support plate; and
side plates,
wherein the plurality of the reagent vessels are integrally connected with each other in parallel by the support plate, and a group of the plurality of the reagent vessels is surrounded by the side plates.

2. The reagent cartridge according to claim 1 further comprising:
an independent reagent vessel instead of at least one of the plurality of the reagent vessels and separately therefrom; and
an engagement portion,
wherein the engagement portion engages the independent reagent vessel in the engagement portion so as to be universally attached and detached, and to be in parallel with the plurality of the reagent vessels.

3. The reagent cartridge according to claim 1 further comprising locked portions universally locked and unlocked by the microorganism detection apparatus.

4. A reagent cartridge for a microorganism detection apparatus, the cartridge comprising:
a plurality of reagent vessels; and
a sealing member,
wherein the plurality of the reagent vessels are integrally connected with each other in parallel by nothing but the sealing member.

5. The reagent cartridge according to claim 4 further comprising side plates, wherein predetermined reagents are instilled in the plurality of the reagent vessels, and openings thereof are closed by the sealing member.

6. The reagent cartridge according to claim 5, wherein the sealing member essentially consists of one sheet member for covering the openings of all of the plurality of the reagent vessels.
